(19) United States
(12) Patent Application Publication
(10) Pub. No.: US 2026/0152492 A1
(43) Pub. Date: Jun. 4, 2026

(54) Carboxamide compounds as a selective EPAC2 activator

(71) Applicant: Celagenex Research (India) Pvt. Ltd, Thane West (IN)

(72) Inventors: Rajaram SAMANT, Thane West (IN); Manoj TONGRA, Jaipur (IN)

(21) Appl. No.: 19/405,069

(22) Filed: Dec. 1, 2025

(30) Foreign Application Priority Data
Nov. 30, 2024 (IN) 202421042585

(51) Int. Cl. C07D 405/12; A61K 31/352

(52) U.S. Cl. CPC C07D 405/12; A61K 31/352

(57) ABSTRACT

The present invention relates to novel carboxamide compounds of Formula-I, which act as potent activators of Exchange Protein Activated by cAMP 2 (EPAC2). The invention further provides a sustainable, one-pot process for the preparation of these EPAC2-activating compounds of Formula-I. Accordingly, the compounds are useful in the treatment and management of EPAC2-associated therapeutic conditions including neurological disorders, synaptic dysfunction, cognitive impairment, and neurodegeneration, as well as disorders linked to impaired cAMP-mediated neuronal signalling.

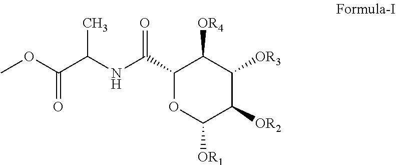

### FIELD OF THE INVENTION

The present invention relates to a novel carboxamide compounds represented by Formula-I as a selective Exchange Protein Activated by cAMP 2 (EPAC2) activator. Particularly, the present invention relates to a novel carboxamide compounds represented by Formula-I and a pharmaceutically acceptable salt thereof which are useful for treating degenerative cervical myelopathy and neurological disorders thereof:

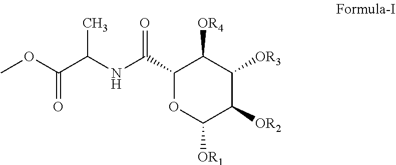

wherein R₁, R₂, R₃ and R₄ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, and a 5 or 6-membered heterocyclic ring having 1 to 4 heteroatoms, wherein the heteroatoms are selected from O, N, S, wherein the 5 or 6 membered heterocyclic ring is,

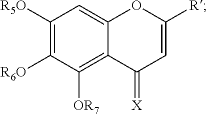

wherein R′ is selected from the group consisting of hydrogen, hydroxyl, halogen, (C₁-C₆) alkyl, (C₂-C₈) alkylene, (C₂-C₈) alkyne and (C₆-C₁₀) aryl;
wherein R₅, R₆ and R₇ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, a 5 or 6-membered heterocyclic ring having 1 to 4 heteroatoms, —C═O, —C═OR₄, —CO₂R₅, —NO₂, —CONR₅R₆, —N(R₅)COR₆; —NR₅R₆, —N(R₅)COR₆, —CN, —CHFCN and CF₂CN; wherein
X is substituted or unsubstituted O, N and S.

### BACKGROUND OF THE INVENTION

Carboxamide compounds have a rich history and are an important class of organic compounds in both biology and chemistry. The term “carboxamide” refers to a group of compounds characterized by the presence of a carboxamide functional group, which is a derivative of carboxylic acids where the hydroxyl group is replaced by an amine group.

In organic chemistry, an amide, also known as an organic amide or a carboxamide, is a compound with the general formula R—C(═O)—NR′R″, where R, R′, and R″ represent any group, typically organyl groups or hydrogen atoms.

Amides, including carboxamides, plays an important role in biochemistry, particularly in the structure of proteins, where they form the peptide bonds that link amino acids together. In the early 19th century, scientist began to understand the structure and properties of amides through the work of pioneering scientists like Justus von Liebig and Friedrich Wohler. Their research laid the groundwork for the synthesis and characterization of amides and carboxamides.

The development of synthetic organic chemistry in the late 19th and early 20th centuries led to the discovery and production of a wide variety of carboxamide compounds. These compounds have been used in numerous applications, from pharmaceuticals to plastics. For instance, the discovery of sulfonamides in the 1930s, which are related to carboxamides, marked the beginning of the antibiotic era. The synthesis of nylon in the 1930s by Wallace Carothers at DuPont involved the polymerization of carboxamide groups, leading to the creation of one of the first synthetic fibers and revolutionizing the textile industry.

Carboxamide compounds have been extensively studied as inhibitors of Phosphodiesterase 4 (PDE4), an enzyme that breaks down cyclic adenosine monophosphate (cAMP), a messenger molecule that plays a crucial role in various physiological processes. PDE4 inhibitors are of significant interest due to their therapeutic potential in treating a range of conditions, including respiratory diseases like chronic obstructive pulmonary disease (COPD) and asthma, as well as psoriasis and other autoimmune disorder. [Int. J. Mol. Sci. 2023, 24, 11518]

Carboxamide derivatives as inhibitors of phosphodiesterase 4 (PDE4) have been researched for their potential therapeutic effects on neurodegenerative diseases, such as Parkinson's disease (PD). PDE4 is an enzyme that degrades cyclic adenosine monophosphate (cAMP), a molecule that plays a crucial role in various biological functions. In the context of neurodegeneration, the dysregulation of PDE4 and cAMP balance can affect neurological processes and contribute to the progression of diseases like PD. [Nongthombam, P. D., Haobam, R. Targeting phosphodiesterase 4 as a potential therapy for Parkinson's disease: a review. Mol Biol Rep 51, 510 (2024).]

There are few literature available in art which discloses PDE4 inhibitors and uses thereof. U.S. Pat. No. 6,740,655B2 describes pyrimidine carboxamides useful as inhibitors of PDE4 isozymes for the treatment of a number of inflammatory, respiratory and allergic diseases and conditions, but especially for the treatment of asthma; chronic obstructive pulmonary disease (COPD) including chronic bronchitis, emphysema, and bronchiectasis; chronic rhinitis; and chronic sinusitis. WO01/57036 and WO03/068235 describe nicotinamide derivatives which are PDE4 inhibitors useful in the treatment of various inflammatory allergic and respiratory diseases and conditions. Carboxamides have been investigated as phosphodiesterase 4 (PDE4) inhibitors in the context of central nervous system (CNS) disorders. The PDE4 inhibitors increase cAMP accumulation and activate protein kinase A (PKA). This signaling pathway plays a crucial role in the development of psychiatric disorders. PDE4 inhibition can lead to anti-inflammatory effects and neuroprotection. [Wang, C., Wang, Z., Li, M., Li, C., Yu, H., Zhou, D., & Chen, Z. (2017). Reducing Neuroinflammation in Psychiatric Disorders: Novel Target of Phosphodiesterase 4 (PDE4) and Developing of the PDE4 Inhibitors.]

The phosphodiesterase 4 (PDE4) inhibitor, rolipram (which readily crosses the blood-brain barrier) overcomes inhibitors of regeneration in myelin in culture and promotes regeneration in vivo. Two weeks after a hemisection lesion at C3/4, with embryonic spinal tissue implanted immediately at the lesion site, a 10-day delivery of rolipram results in considerable axon regrowth into the transplant and a significant improvement in motor function. [Nikulina et al, PNAS, Jun. 8, 2004, vol. 101, no. 23,8789]

The systemic delivery of a PDE4 inhibitor leads to enhanced differentiation of OPCs (oligodendrocyte progenitor cells) within focal areas of toxin-induced demyelination and a consequent acceleration of remyelination. [EMBO Mol Med (2013) 5, 1918-1934]

The recent study which investigates the disease-modulating activity of the phosphodiesterase 3/phosphodiesterase 4 inhibitor (Ibudilast) as an adjuvant to surgical decompression in DCM. This study is significant because it explores the potential of a PDE4 inhibitor to benefit recovery in DCM, a condition that can lead to progressive spinal cord dysfunction due to compression. [Davies B, et al. BMJ Open 2023]

Carboxamide compounds continue to be a focus of research and development due to their versatility and the broad range of biological activities they exhibit. They are key components in the design of new drugs and materials, demonstrating the enduring importance of this class of compounds in science and industry.

The past research and development are restricted to use of carboxamide compounds as PDE (phosphodiesterase) inhibitors in the treatments for neurological diseases. Particularly, the use of PDE inhibitors in the treatment of degenerative cervical myelopathy (DCM) was an area of interest. However, PDE activation leads to direct activation of cAMP would likely to cause several adverse effects on cellular functions. Uncontrolled activation of the cAMP-dependent pathway can lead to hyper-proliferation of cells, which may contribute to the development and progression of cancer.

High levels of cAMP also led to activation of protein kinase A (PKA). The activated PKA then phosphorylate various target proteins, including PDEs. PKA-mediated phosphorylation again increases PDE activity, leading to more efficient degradation of cAMP. Hence, Using PDE inhibitor and increasing cAMP level is deemed to be a short-term treatment option and may not be considered as effective in neurological disorders such as DCM.

Hence, there is a huge need of good medication which is independent to PDE/cAMP pathway and provides long term effect in neurological disorders. The recent studies suggest that a targeted activation of EPAC2 (Exchange protein directly activated by cAMP 2) might be necessary to see significant improvements in functional recovery from neurological disorders and limiting possible side effects that may occur from a broader cAMP boosting treatment. EPAC2 activation promotes axonal regeneration like the administration of upstream signalling molecule i.e. cAMP. EPAC2 agonists or activator increase neurite outgrowth, induce attractive growth cone turning and mitigate axonal growth inhibition. EPAC2 also activates CREB, which is sufficient to induce the regeneration of sensory axons after a neurological injury. [The Journal of Neuroscience, Mar. 11, 2020 •40(11):2184-2185]

Therefore, the inventors of present invention have developed novel chemical entity of carboxamide compound which is selective EPAC2 activator and generally safe for administration with improved bioavailability and reduced side effects.

### OBJECTIVES OF THE INVENTION

The primary objective of the present invention is to provide a novel therapeutically active carboxamide compounds represented by Formula-I.

Yet another objective of the present invention is to provide a novel therapeutically active carboxamide compounds represented by Formula-I which is a selective EPAC2 activator.

Another objective of the present invention is to provide cost-effective, side-effect-free carboxamide compounds represented by Formula-I.

Another objective of the present invention is to provide an improved and commercially viable process for preparation of novel carboxamide compounds represented by Formula-I.

Another objective of the present invention is to provide therapeutically active carboxamide compounds for treating neurological disorders.

### SUMMARY OF THE INVENTION

To meet the above objectives, the inventors of the instant invention carried out thorough experiments to establish significant effects of the carboxamide compounds that ameliorate therapeutic efficacy in the treatment of neurological disorders and disabilities.

In a particular aspect, the present invention relates to novel therapeutically active carboxamide compounds represented by Formula-I and a pharmaceutically acceptable salt thereof.

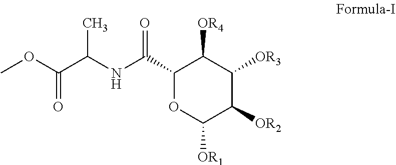

In another aspect, the present invention provides novel carboxamide compounds represented by Formula-I with high bioavailability.

In another aspect, the present invention provides a novel carboxamide compounds represented by Formula-I prepared by a commercially viable and non-hazardous process.

In another aspect, the present invention provides therapeutically effective, non-toxic and safe novel carboxamide compounds with no major side effects.

In a further aspect, the present invention provides pharmaceutical composition comprising Formula I compound along with pharmaceutically acceptable excipients/carriers useful in the treatment of neurological, neurovascular, cerebrovascular, and metabolic disorders associated with endothelial dysfunction and advanced glycation stress.

### BRIEF DESCRIPTION OF THE FIGURES

FIG. 1 illustrates NMR data of Carboxamide Compound of Formula-Ia.

FIG. 2 illustrates NMR data of Carboxamide Compound of Formula-lb.

FIG. 3 illustrates NMR data of Compound of Formula-Ic.

FIG. 4 illustrates the HPLC data of compound of Formula Ia, RT-methyl alaninate at 2.1 min, 7-D-Glucuronic acid-5,6-dihydroxyflavone-9.5 min.

FIG. 5 illustrates the EPAC2 expression of control, non-conjugated-, Compound of Formula Ia, Compound of Formula Ib and Compound of Formula Ic.

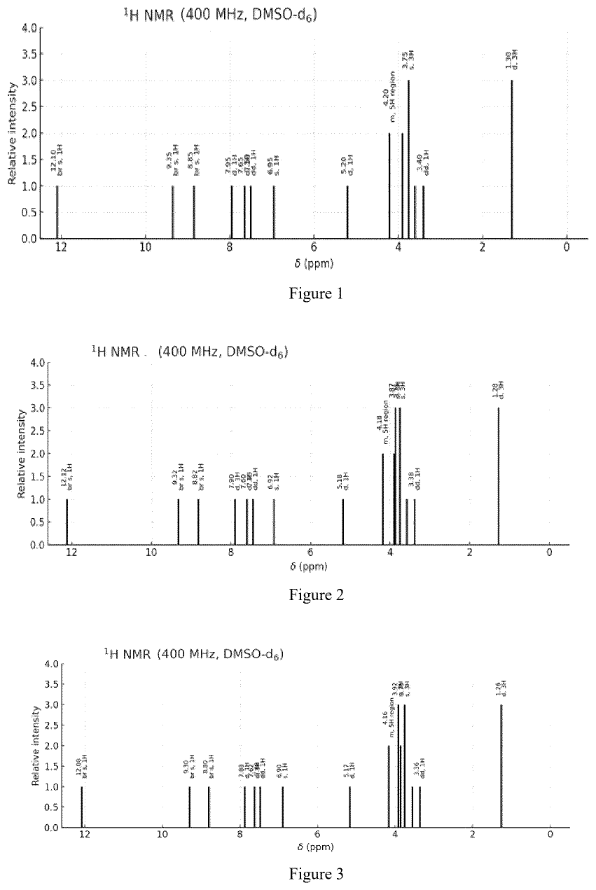

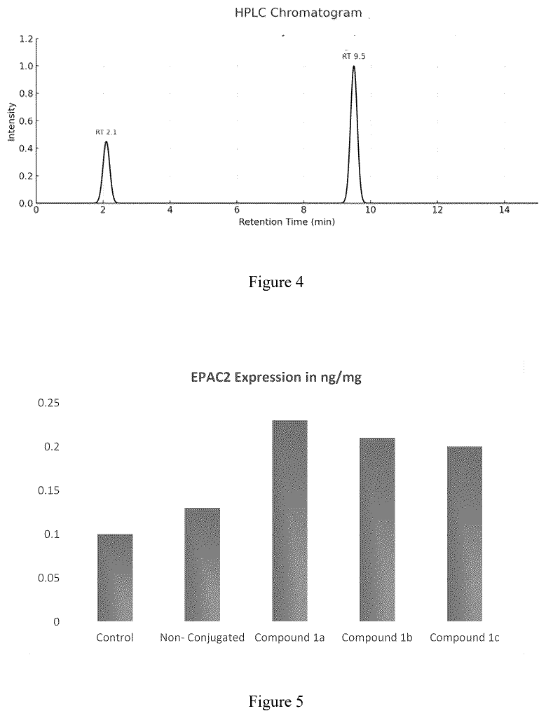

### DETAILED DESCRIPTION OF THE INVENTION

For the purpose of promoting an understanding of the principles of the invention, reference will now be made to the embodiments, in the specific language will be used to describe the same. It will nevertheless be understood that no limitation of the scope of the invention is thereby intended, such alterations and further modifications in the illustrated composition, and such further applications of the principles of the invention as illustrated therein being contemplated as would normally occur to one skilled in the art to which the invention relates.

Unless otherwise defined, all technical and scientific terms used herein have the same meaning as commonly understood by one of ordinary skilled in the art to which this invention belongs. The composition, methods, and examples provided herein are illustrative only and not intended to be limiting.

It is further to be understood that all terminology used herein is for the purpose of describing particular embodiment only and is not intended to be limiting in any manner or scope. Unless defined otherwise, all technical and scientific expressions used herein have the same meaning as commonly understood by one of ordinary skill in the art to which embodiments of the invention pertain.

In describing and claiming the embodiments of the present invention, the following terminology will be used in accordance with the definitions set out below which are known in the state of art. The singular forms “a,” “an,” and “the” include plural reference unless the context clearly dictates otherwise. Also, the term ‘composition’ does not limit the scope of the invention for multiple compositions that can be illustrated for best mode of the invention.

Also, the term ‘composition’ does not limit the scope of the invention for multiple compositions that can be illustrated for best mode of the invention.

The term “composition” and “formulation” as used herein can be interchangeably used.

The term “pharmaceutically/nutraceutically acceptable salt,” as used herein, represents those salts which are within the scope of sound medical judgment, suitable for use in contact with the tissues of humans and animals without undue toxicity, irritation, allergic response and the like and are commensurate with a reasonable benefit/risk ratio.

Particularly the term “pharmaceutically-acceptable salts” refers to the relatively non-toxic, inorganic and organic acid addition salts of compounds, amino acid salt, sugar-based salt, alkali or alkaline earth metal salts, as well as solvates, co-crystals, polymorphs and the like of the salts. All modifications and substitutions that come within the meaning of the description and the range of their legal equivalents are to be embraced within their scope. A description using the transition “comprising” allows the inclusion of other elements to be within the scope of the invention.

In a preferred embodiment, the present invention provides novel carboxamide compounds of Formula-I, which act as potent activators of Exchange Protein Activated by cAMP 2 (EPAC2).

In a preferred embodiment, the present invention provides therapeutically significant novel chemical entity of carboxamide of Formula-I in stable form.

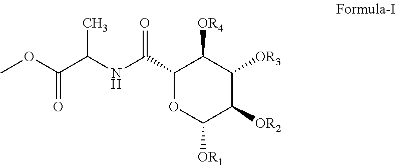

wherein R₁, R₂, R₃ and R₄ is similar or independently is selected from the group consisting of hydrogen, hydroxyl, halogen, (C₁-C₆) alkyl, (C₂-C₅) alkylene, (C₂-C₈) alkyne, (C₃-C₅) cycloalkyl, (C₁-C₆) alkoxy, (C₆-C₁₀) aryl, and a 5 or 6-membered heterocyclic ring having 1 to 4 heteroatoms, wherein the heteroatoms are selected from O, N, S, wherein the 5 or 6-membered heterocyclic ring is,

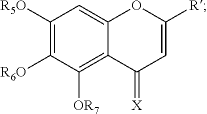

wherein R′ is selected from the group consisting of hydrogen, hydroxyl, halogen, (C₁-C₆) alkyl, (C₂-C₈) alkylene, (C₂-C₈) alkyne and (C₆-C₁₀) aryl;
wherein R₅, R₆ and R₇ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, a 5 or 6-membered heterocyclic ring having 1 to 4 heteroatoms, —C═O, —C═OR₄, —CO₂R₅, —NO₂, —CONR₅R₆, —N(R₅) COR₆; —NR₅R₆, —N(R₅) COR₆, —CN, —CHFCN and CF₂CN; wherein
X is substituted or unsubstituted, O, N and S.

In another embodiment, the present invention provides salts of compounds of Formula I, wherein salt is selected from but not limited to the group consisting of amino acid salts, chloride, sulfate, phosphate, diphosphate, bromide, nitrate salts, hydrochloride, hydrobromide, malate, maleate, fumarate, tartrate, succinate, citrate, taurate, threonate, acetate, lactate, methanesulfonate, benzoate, ascorbate, sulfonate, salicylate as well as sodium, potassium, calcium, lithium, magnesium and ammonium (including substituted ammonium).

In yet another embodiment, the present invention provides an improved process for the preparation of novel carboxamide compounds of formula I and pharmaceutically acceptable salts thereof.

In some embodiment, the present invention provides novel carboxamide compounds of Formula I for treating degenerative cervical myelopathy and neurological disorders thereof.

In another preferred embodiment, the invention provides compounds of formula I, wherein the substituent salt is selected from but not limited to the group consisting of amino acid salts, chloride, sulfate, phosphate, diphosphate, bromide, nitrate salts, hydrochloride, hydrobromide, malate, maleate, fumarate, tartrate, succinate, citrate, taurate, threonate, acetate, lactate, methanesulfonate, benzoate, ascorbate, sulfonate, salicylate as well as, sodium, potassium, calcium, lithium, magnesium and ammonium (including substituted ammonium).

In yet another preferred embodiment, the novel chemical entity of carboxamide of Formula-I is selected from the group consisting of but not limited to methyl 2-((2S,3S,4S,5R,6S)-6-((5,6-dihydroxy-4-oxo-2-phenyl-4H-chromen-7-yl)oxy)-3,4,5-trihydroxytetrahydro-2H-pyran-2-carboxamido)propanoate; methyl 2-((2S,3S,4S,5R,6S)-6-((5-methoxy,6-hydroxy-4-oxo-2-phenyl-4H-chromen-7-yl)oxy)-3,4,5-trihydroxytetrahydro-2H-pyran-2-carboxamido)propanoate; methyl 2-((2S,3S,4S,5R,6S)-6-((5-hydroxy,6-methoxy-4-oxo-2-phenyl-4H-chromen-7-yl)oxy)-3,4,5-trihydroxytetrahydro-2H-pyran-2-carboxamido) propanoate and a combination thereof.

In another embodiment, the free base of compounds of Formula I is optionally stabilized with pharmaceutically acceptable salts such as, chloride, bromide, hydrochloride, hydrobromide, hydrate, sodium, magnesium, carbonate, calcium and like thereof.

In another embodiment, the presently improved in situ process provides compounds of Formula I with high purity and yield, wherein the HPLC purity is not less than 90% after drying.

In another embodiment, the presently improved in situ process provides compounds of Formula-I with high purity and yield, wherein the yield and purity is not less than 70%. Preferably, the practical yield is in the range of 75% to 100%.

In yet another embodiment, the present invention provides novel carboxamide compounds for treatment of degenerative cervical myelopathy and neurological disabilities.

Further, the present novel compounds are useful in the treatment of degenerative cervical myelopathy selected from but not limited to cervical spondylotic myelopathy (CSM), degenerative disc disease (DDD), and ossification of ligaments like the ossification of the posterior longitudinal ligament (OPLL) and the ossification of the ligamentum flavum (OLF); and neurological disabilities are selected from but not limited to parkinson's disease, alzheimer's disease, ataxia, migraine, stroke, encephalitis and like thereof.

The bioactive flavonoid compounds of Formula I has demonstrated therapeutic potential in Acute Kidney Injury (AKI) and renal fibrosis due to its antioxidant, anti-inflammatory, antifibrotic, and apoptosis-modulating properties. It helps to prevent progression to Chronic Kidney.

In another embodiment, the present invention provides novel carboxamide compounds which are selective EPAC2 activator.

In another preferred embodiment, the present invention provides novel carboxamide compounds which selectively increases mitochondrial cAMP level and mediates neuronal survival.

In yet another embodiment, the present invention provides novel carboxamide compounds for treatment of degenerative cervical myelopathy and neurological disabilities.

In another preferred embodiment, the present invention provides novel therapeutically active ingredient of the carboxamide compounds represented by Formula Ia, Formula Ib and Formula Ic.

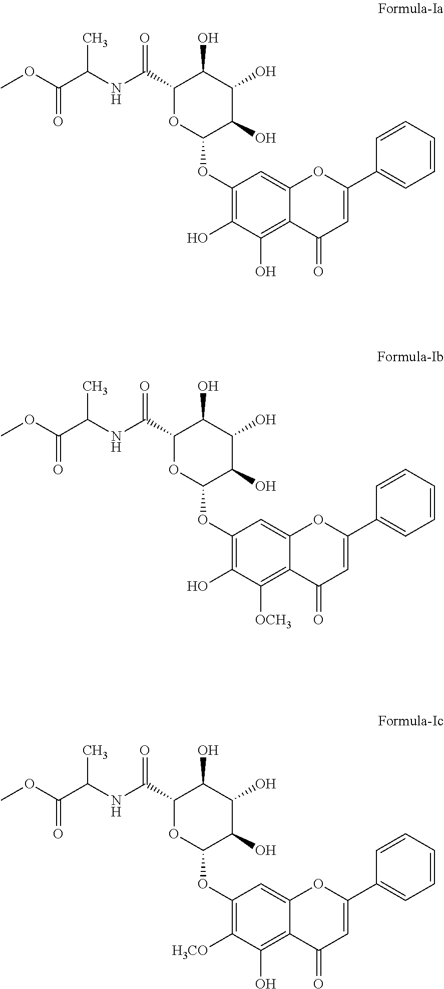 [SMILES: COC(=O)C(C)NC(=O)[C@H]1O[C@@H](Oc2cc3oc(-c4ccccc4)cc(=O)c3c(O)c2O)[C@H](O)[C@@H](O)[C@@H]1O ; COC(=O)C(C)NC(=O)[C@H]1O[C@@H](Oc2cc3oc(-c4ccccc4)cc(=O)c3c(OC)c2O)[C@H](O)[C@@H](O)[C@@H]1O ; COC(=O)C(C)NC(=O)[C@H]1O[C@@H](Oc2cc3oc(-c4ccccc4)cc(=O)c3c(O)c2OC)[C@H](O)[C@@H](O)[C@@H]1O]

Further, the present novel compounds are useful in the treatment of degenerative cervical myelopathy and neurological disorders thereof. Particularly, the present novel compounds are a selective EPAC2 activators and plays a crucial role in neuronal regeneration and plasticity. Additionally, the present novel compounds selectively increase mitochondrial cAMP (where PDE inhibitors only increases cytosolic cAMP) level which improves mitochondrial stability and function. Improved mitochondrial stability and function provides an adequate energy supply for neurons and thus mediates neuronal survival.

In another embodiment, the present invention further provides a sustainable, one-pot process for the preparation of these EPAC2-activating compounds of Formula-I.

In yet another embodiment, the present invention provides a one-pot process for the preparation of novel carboxamide compounds of Formula I, comprising the steps of:
(a) dissolving a 7-D-Glucuronic acid-5,6-dihydroxyflavone compound in a hydroalcoholic solvent;
(b) activating a carboxyl group of the 7-D-Glucuronic acid-5,6-dihydroxyflavone compound in-situ using a carbodiimide coupling reagent and N-hydroxysuccinimide at a temperature below of 5° C. to form an activated intermediate;
(c) reacting the activated intermediate with methyl DL alaninate in the presence of an organic base to form a product;
(d) precipitating the product by solvent adjustment; and
(e) isolating the product carboxamide compound of Formula I having HPLC purity of ≥98%.

In another embodiment of the present invention the product is a carboxamide compound 7-D-Glucuronic acid-5,6-dihydroxyflavone of formula 1.

In another embodiment, the process comprising a hydroalcoholic solvent comprises a C₁-C₄ alkanol and water, particularly selected from ethanol-water, methanol-water, isopropanol-water, n-propanol-water, or mixtures thereof. In the process the organic base is selected from triethylamine, N,N-diisopropylethylamine, N-methylmorpholine, pyridine, sodium, potassium hydroxide, ammonium hydroxide, ammonia, primary or secondary amine and mixtures thereof. The reduced temperature for coupling reaction is maintained below 5° C. The one pot process wherein the reaction is conducted at 20-35° C. for 4-6 hours. The isolated yield of the carboxamide compounds varies between 78-90%.

In one embodiment, the present invention relates to a pharmaceutical composition comprising a methyl alaninate conjugate of Formula-I as an active therapeutic agent and pharmaceutically acceptable salts, solvates, polymorphs, hydrates, stereoisomers, or prodrugs thereof.

In yet another embodiment, the methyl alaninate conjugate of Formula-I enhances pharmacokinetic properties including increased membrane permeability, improved oral bioavailability, and enhanced metabolic stability compared to unmodified compound of Formula-I.

In another embodiment, the pharmaceutical composition comprising the methyl alaninate conjugate of Formula-I is formulated for the prevention or treatment of diseases associated with oxidative stress, neuroinflammation, metabolic disorders, or hormonal dysregulation.

In another embodiment, the composition comprising the methyl alaninate conjugate of Formula I is administered to a subject in need thereof via oral, parenteral, sublingual, transdermal, or intranasal route.

In a further embodiment, the pharmaceutical composition optionally comprises one or more additional therapeutic agents, excipients, carriers, stabilizers, or penetration enhancers suitable for pharmaceutical use.

In one more embodiment, the compounds of Formula-I are useful in the treatment and management of EPAC2-associated therapeutic conditions including neurological disorders, synaptic dysfunction, cognitive impairment, and neurodegeneration, as well as disorders linked to impaired cAMP-mediated neuronal signaling.

In some more embodiment, the methyl alaninate conjugate of Formula-I demonstrates enhanced EPAC2 activation, thereby improving intracellular cAMP signaling pathways useful in conditions such as male infertility, impaired HPG axis function, diabetes, or cognitive impairment.

The term ‘subject in need thereof’ pertains to a subject preferably mammal, more preferably a human suffering or suspected with degenerative cervical myelopathy.

In the context of the present invention, the term “treatment” refers to alleviate, mitigate, prophylaxis, attenuate, manage, regulate, modulate, control, minimize, lessen, decrease, down regulate, up regulate, moderate, inhibit, restore, suppress, limit, block, decrease, prevent, inhibit, stabilize, ameliorate, cure, heal metabolic or nervous system related disorders observed in the patient. Notably, the present novel carboxamide moieties are non-hazardous, non-toxic, and safe for human consumption without any severe adverse effects, therefore the present medicinal composition can also be used as preventive therapy, adjuvant therapy, add-on therapy, combination, adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the invention are formulated in geometric or, enantiomeric or stereoisomeric forms.

In general, all physical forms are of use in the methods contemplated by the present invention and are intended to be within the scope of the invention.

Compound or pharmaceutically acceptable salts includes, hydrates, polymorphs, solvates, enantiomers or racemates. Some of the crystalline forms of the compound exist as polymorphs and as such are intended to be included in the present disclosure. In addition, some of the compounds may form solvates with water (i.e., hydrates) or common organic solvents, and such solvates are intended to be encompassed by some embodiments.

In one of the embodiments, the present invention provides medicinal composition comprising novel carboxamide compounds of Formula-I present in effective amount along with pharmaceutically acceptable excipients.

As used herein, the term “pharmaceutically acceptable carriers, diluents or excipients” is purported to mean, without limitation, any adjuvant, carrier, excipient, sweetening agent, diluents, preservative, dye/colorant, flavour enhancer, surfactant, wetting agent, dispersing agent, suspending agent, stabilizer, isotonic agent, solvent, emulsifier, or encapsulating agent, encapsulating polymeric delivery systems or polyethylene glycol matrix, which is acceptable for use in the subject, preferably humans. Excipients also include, for example: antiadherents, antioxidants, binders, coating agents, compression aids, disintegrants, dyes (colours), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, or waters of hydration, salts.

In another embodiment, the present invention relates to medicinal composition of carboxamide compounds of Formula-I prepared in a manner well known in the pharmaceutical art and administered by a variety of routes, depending upon whether local or systemic treatment is desired and upon the area to be treated.

The preferable route of administration includes but is not limited to sublingual, rectal, topical, parenteral, nasal, or oral.

In another embodiment, the compounds of Formula I of the present invention are non-toxic, cost effective and provide safeguard against problems associated with degenerative cervical myelopathy and neurological disorders without any adverse effect.

In some embodiments, the compounds of Formula I can be formulated in medicaments by using pharmaceutically acceptable excipients. The pharmaceutically acceptable excipients are selected from the group consisting of, but not limited to, diluents or fillers, binders, disintegrants, lubricants, glidants, solubilizing agents, surfactants, stabilizers, colours, flavouring agents, sweeteners, plasticizers, a coating agent, colours, flavouring agents, sweeteners, pH adjusting agents, buffers, antioxidants other additives or mixtures thereof. The amount of excipient(s) employed will depend upon how much active agent is to be used. One excipient can perform more than one function.

The pharmaceutical composition further comprises pharmaceutically acceptable excipient. The pharmaceutically acceptable excipients are selected from the group consisting of, but not limited to, diluents or fillers, binders, disintegrants, lubricants, glidants, solubilizing agents or surfactants, solvents, stabilizers, sweeteners, flavouring agents, plasticizers, antioxidants coating agents and other additives or mixtures thereof.

In another embodiment of the present invention, the diluents are selected from but not limited to, starches, hydrolyzed starches, partially pregelatinized starches, anhydrous lactose, cellulose powder, lactose monohydrate, sugar alcohols such as sorbitol, xylitol and mannitol, Maltitol, Lactitol, silicified microcrystalline cellulose, ammonium alginate, calcium carbonate, calcium lactate, dicalcium phosphate, dibasic calcium phosphate (anhydrous/dibasic dehydrate/tribasic), calcium silicate, calcium sulphate, cellulose acetate, corn starch, pregelatinized starch, Sodium Starch Glycolate, dextrin, β-cyclodextrin, dextrates, dextrose, erythritol, ethyl cellulose, fructose, fumaric acid, glyceryl palmitostearate, magnesium carbonate, magnesium oxide, maltodextrin, maltose, medium-chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or mixtures thereof.

In some embodiment of the present invention, the diluent or filler in the composition/formulation is present in a range of 1% to 30% by weight of the total composition/formulation.

In yet another embodiment of the present invention, the binders are selected from but not limited to, disaccharides such as sucrose, lactose, polysaccharides and their derivatives like starches, cellulose, or modified cellulose such as microcrystalline cellulose and cellulose ethers such as hydroxypropyl cellulose(HPC); hydroxypropyl methyl cellulose (HPMC); sugar alcohols such as xylitol, sorbitol, or mannitol, Lactitol, Maltitol; protein like gelatin; synthetic polymers such as polyvinylpyrrolidone (PVP), polyethylene glycol (PEG), starch, acacia, agar, alginic acid, calcium carbonate, calcium lactate, carbomers, carboxymethylcellulose sodium, carrageenan, cellulose acetate phthalate, chitosan, co-povidone, corn starch, pregelatinized starch, Sodium Starch Glycolate, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid glucose, hypromellose, magnesium aluminum silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants, wax or mixtures thereof.

In further embodiment of the present invention, the binder in the composition/formulation is present in a range of 0.1 to 25% by weight of the composition/formulation.

In another embodiment of the present invention, the disintegrants are selected from but not limited to, Polyvinylpolypyrrolidone (polyvinyl polypyrrolidone, PVPP, crospovidone, crospolividone or E1202) is a highly cross-linked modification of polyvinylpyrrolidone (PVP), calcium carbonate, sodium starch glycolate, croscarmellose sodium, microcrystalline cellulose, low-substituted hydroxypropyl cellulose (L-HPC), mannitol, colloidal silicon dioxide, hydrated 18 silica and/or hypromellose, maize starch, salts of carboxy methyl cellulose, alginic acid, sodium alginate, guar gum or mixtures thereof.

In further embodiment of the present invention, the disintegrants in the composition/formulation are present in a range of 0.1 to 10% by weight of the composition/formulation.

In another embodiment of the present invention, the lubricants are selected from but not limited to, magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or mixtures thereof.

In some embodiment of the present invention, the lubricant in the composition/formulation is present in a range of 0.1% to 10.0% by weight of the total composition/formulation.

In some embodiment of the present invention, the glidants are selected from but not limited to, colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or mixtures thereof.

In another embodiment of the present invention, the glidant in the composition/formulation is present in a range of 0.1% to 5.0% by weight of the total composition/formulation.

In another embodiment of the present invention, the solubilizing agents or surfactants are selected from but not limited to, polysorbates such as polysorbate 80, polysorbate 60, polysorbate 20; sodium lauryl sulphate, anionic emulsifying wax, nonionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, benzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, 15 methylparaben, propylparaben, sorbic acid or mixtures thereof.

In another embodiment of the present invention, the amount of solubilizing agent or surfactant in composition/formulation ranges from 0.1% to 10% by weight of the composition/formulation.

In a preferred embodiment of the present invention, the solubilizing agent or surfactant is present in a range of 0.1% to 5.0% by weight of the total composition/formulation.

In some embodiment of the present invention, the solvents are selected from but not limited to, water, alcohol, isopropyl alcohol, propylene glycol, mineral oil, benzyl alcohol, benzyl benzoate, flavoured glycol, carbon dioxide, castor oil, corn oil (maize), cottonseed oil, dimethyl ether, albumin, dimethylacetamide, ethyl acetate, ethyl lactate, medium-chain triglycerides, methyl lactate, olive oil, peanut oil, polyethylene glycol, polyoxyl, propylene carbonate, pyrrolidone, safflower oil, sesame oil, soybean oil, sunflower oil, water-miscible solvents, organic polar or non-polar solvents or mixtures thereof.

In a preferred embodiment of the present invention, the solvent in the composition/formulation is used in a quantity sufficient to make the weight of the composition/formulation 100% by weight.

In some embodiment of the present invention, the stabilizers are selected from but not limited to, the group consisting of alginate, agar, carrageen, gelatin, guar gum, gum arabic, locust bean gum, pectin, starch, xanthan gum, trehalose or mixtures thereof.

In some embodiment of the present invention, the stabilizer in the composition/formulation is present in a range of 0.1% to 5.0% by weight of the total composition/formulation.

In some embodiment of the present invention, the sweeteners are selected from but not limited to sorbitol, sucrose, dextrose, fructose, mannitol, xylitol, aspartame, stevia extract, glycyrrhiza, saccharine, saccharine sodium, acesulfame, sucralose, dipotassium glycyrrhizinate, galactose, fructose, high fructose corn syrup, se, sucrose, sugar, maltose, partially hydrolyzed starch, corn syrup solids, D-tryptophan, erythritol, fructose, galactose, glycerol, glycyrrhizin, glucose, isomalt, xylitol, xylose, lactitol, lactose, levulose, maltitol, maltodextrin, maltol, maltose, corn syrup, neohesperidin dihydrochalcone, neotame, sodium saccharin, siclamate, tagatose, taumatin, trehalose or mixtures thereof.

In an embodiment the sweeteners are present in the range of 0.1% to 5% by the weight of the total composition.

In some embodiment of the present invention, the flavouring agent/flavourant is selected from a group comprising: volatile oil, aldehyde, ginger oil, double mint, peppermint oil, menthol, lemongrass oil, fruit, citrus, cherry and a combination thereof.

In an embodiment the flavouring agent/flavourant are present in the range of 0.1% to 5% by the weight of the total composition.

In some embodiment of the invention, the plasticizers are added to coating formulations are selected from but not limited to, the group propylene glycol, polyethylene glycol, glycerol, glyceryl triacetate (triacetin), triethyl citrate, acetyl triethyl citrate, diethyl phthalate, actetylated monoglycerides, castor oil, mineral oil or mixtures thereof.

In some embodiment of the present invention, the plasticizer in the composition/formulation is present in a range of 0.1% to 5.0% by weight of the total composition/formulation.

In some embodiment of the present invention, the antioxidants are selected from but not limited to, Ascorbic acid, Citric acid, Sodium Ascorbate, Ascorbyl Palmitate, odium bisulfite, sodium sulfite, sodium thiosulfate, sodium metabisulfite, Butyl hydroxyanisole (BHA), Butylated Hydroxytoluene (BHT) or mixtures thereof.

In an embodiment of the present invention, the antioxidants are present in the range of 0.1% to 10% by the weight of the total composition.

Coating agents such as synthetic polymers, shellac, corn protein (zein) or other polysaccharides, gelatin, fatty acids, waxes, shellac, plastics, and plant fibers or mixtures thereof.

In an embodiment of the present invention, the synthetic polymers are present in the range of 0.1% to 5% by the weight of the total composition.

The additional additives include but not limited to, a polymer, a coating agent, a plasticizer, a sweetener, and a flavour, a preservative, a colorant, a surfactant, pH adjusting agents, buffers and other excipients.

In a preferred embodiment of the present invention, the additives are used in a range of 1 to 20% w/w of unit dose.

In yet another embodiment, the present invention provides a pharmaceutical composition comprising compounds of Formula I along with pharmaceutical excipients, wherein the pharmaceutical excipients are selected from a diluent, a binder, a lubricant, a glidant, an additive, a surfactant, a stabilizer, antioxidants or mixtures thereof.

In a preferred embodiment, the diluent is present in a range of 1 to 30%; the binder is present in a range of 0.1 to 25%; the disintegrant is present in a range of 0.1 to 10%; the lubricant is present in a range of 0.1 to 10.0%; the glidant is present in a range of 0.1 to 5.0%; the solubilizing agent or surfactant is present in a range of 0.1 to 5.0%; the stabilizer is present in a range of 0.1 to 5.0%; the plasticizer is present in a range of 0.1 to 5.0%; the antioxidant is present in a range of 0.1 to 10%; the additive is present in a range of 1 to 20% by weight of total composition.

In an embodiment of the present invention, the pharmaceutical composition is characterized for treating or preventing neurological disorders associated with impaired EPAC2 activity, wherein the neurological disorders comprise cognitive impairment, synaptic dysfunction, Alzheimer's disease, Parkinson's disease, or neurodegeneration.

In some embodiment, the pharmaceutical compositions of the carboxamide compound of formula I are administered to a subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray.

In a further embodiment, the pharmaceutical composition of the present invention is formulated in the form of age-appropriate paediatric oral dosage forms such as syrup, inhalation, spray minitablets, chewable formulations, orodispersible films, and orodispersible tablets.

In yet another embodiment, the present invention provides a solid formulation adapted for oral administration to a mammal, particularly human, along with pharmaceutically acceptable excipients for treating or preventing a degenerative cervical myelopathy and neurological disorders.

The magnitude of a prophylactic or therapeutic dose typically varies with the nature and severity of the condition to be treated and the route of administration. The dose and perhaps the dose frequency will also vary according to the age, body weight, and response of the individual patient.

In general, the total daily dose (in single or divided doses) ranges from about 0.1 mg per day to about 5000 mg per day, preferably about 1 mg per day to about 1000 mg per day.

In some embodiments, the total daily dose can be administered in the range of about 1 mg to about 3000 mg per day, and preferably about 1 mg to about 1000 mg per day.

The use of any and all examples, or exemplary language (e.g., such as) provided herein, is intended merely to better illuminate the invention and does not pose a limitation on the scope of the invention unless otherwise claimed.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The invention may be further be illustrated by the following examples, which are for illustrative purposes only and should not be construed as limiting the scope ofthe invention in anyway. The present disclosure is therefore to be considered as in all respects illustrative and not restrictive, the scope of the invention being indicated by the appended claims and examples, and all changes or alterations which come within the ambit of equivalency are intended to be encompassed therein.

### EXAMPLES

### Example 1

TABLE-1
Purity and Yield of Compounds of Formula-I

| | | % | % |
|---|---|---|---|
| Compounds | Compounds Of Formula-I | Purity | Yield |
| Ia | 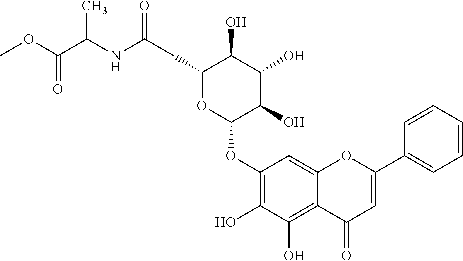 [SMILES: COC(=O)C(C)NC(=O)C[C@H]1O[C@@H](Oc2cc3oc(-c4ccccc4)cc(=O)c3c(O)c2O)[C@H](O)[C@@H](O)[C@@H]1O] | 96 | 88 |
| | Methyl 2-((2S,3S,4S,5R,6S)-6-((5,6-dihydroxy-4-oxo-2-phenyl-4H-chromen- | | |
| | 7-yl)oxy)-3,4,5-trihydroxytetrahydro-2H-pyran-2-carboxamido)propanoate; | | |
| Ib | 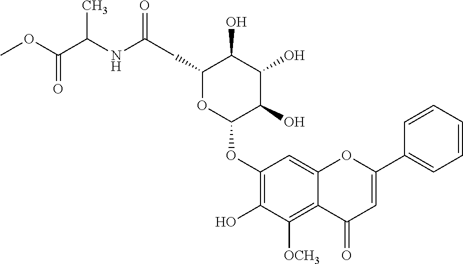 [SMILES: COC(=O)C(C)NC(=O)C[C@H]1O[C@@H](Oc2cc3oc(-c4ccccc4)cc(=O)c3c(OC)c2O)[C@H](O)[C@@H](O)[C@@H]1O] | 95 | 85 |
| | Methyl 2-((2S,3S,4S,5R,6S)-6-((5-methoxy,6-hydroxy-4-oxo-2-phenyl-4H- | | |
| | chromen-7-yl)oxy)-3,4,5-trihydroxytetrahydro-2H-pyran-2-carboxamido) | | |
| | propanoate; | | |
| Ic | 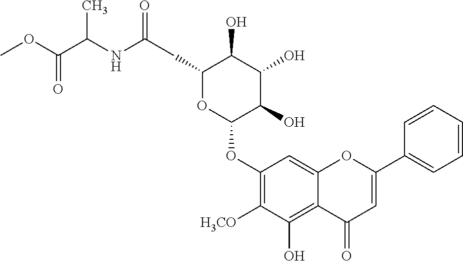 [SMILES: COC(=O)C(C)NC(=O)C[C@H]1O[C@@H](Oc2cc3oc(-c4ccccc4)cc(=O)c3c(O)c2OC)[C@H](O)[C@@H](O)[C@@H]1O] | 94 | 80 |
| | Methyl 2-((2S,3S,4S,5R,6S)-6-((5-hydroxy,6-methoxy-4-oxo-2-phenyl-4H- | | |
| | chromen-7-yl)oxy)-3,4,5-trihydroxytetrahydro-2H-pyran-2- | | |
| | carboxamido)propanoate or combination thereof. | | |

### Compound of Formula 1

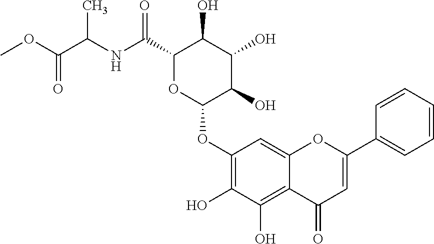 [SMILES: COC(=O)C(C)NC(=O)[C@H]1O[C@@H](Oc2cc3oc(-c4ccccc4)cc(=O)c3c(O)c2O)[C@H](O)[C@@H](O)[C@@H]1O]

### Process for Preparation of Compound of Formula-Ia

### Methyl 2-((2S,3S,4S,5R,6S)-6-((5,6-dihydroxy-4-oxo-2-phenyl-4H-chromen-7-yl)oxy)-3,4,5-trihydroxytetrahydro-2H-pyran-2-carboxamido)propanoate

7-D-Glucuronic acid-5,6-dihydroxyflavone (1.00 mmol, 446.36 mg) was dissolved in ethanol-water (1:1 v/v, total 20.0 mL; EtOH 10.0 mL+H₂O 10.0 mL, final substrate concentration 0.05 M) under gentle heating (30-35° C.) to afford a clear dispersion, and the mixture was cooled to 0-5° C. in an ice bath. To the cooled solution were added N-hydroxy-succinimide (NHS, 1.20 mmol, 138.1 mg) and 1-ethyl-3-(3-dimethylaminopropyl) carbodiimide hydrochloride (EDC HCl, 1.50 mmol, 287.6 mg) as solid portions while maintaining 0-5° C.; the activation period was 30-45 min with magnetic stirring. Methyl alaninate (free base, 1.50 mmol, 154.7 mg) was then introduced as a solution in 2.0 mL EtOH, followed by addition of DIPEA (2.00 mmol, 0.348 mL) dropwise at 0-5° C., and the reaction mixture was allowed to warm to ambient temperature (20-25° C.) and stirred for 4-6 h (monitoring conversion by HPLC). The reaction was quenched by addition of chilled water (30 mL), and the crude mixture was filtered to remove insoluble urea by-products; the product was precipitated by adjusting the ethanol-water ratio (addition of water to achieve EtOH:H₂O 1:3 v/v), collected by vacuum filtration, washed with cold water (2×10 mL) and cold ethanol (1×10 mL), and dried under reduced pressure at 40° C. to afford Compound of Formula Ia (82-86% isolated yield) with HPLC purity ≥98.0%. Identical reaction conditions and stoichiometries (per 1.00 mmol starting material basis) were applied to synthesize Compound of Formula Ib and Compound of Formula Ic (each prepared from 7-D-Glucuronic acid-5,6-dihydroxyflavone precursor, which were isolated in 78-84% and 80-85% yields, respectively, each demonstrating HPLC purity ≥98.5% after the same precipitation and wash procedure.

Purity was confirmed by HPLC (same method) and by ¹H NMR (400 MHz, DMSO-d₆) and HRMS (ESI) analysis.

Drying and isolation steps were carried out under reduced pressure (≤20 mmHg) at ≤40° C. to avoid thermal degradation; all steps were performed using ethanol where specified and with standard inert-atmosphere practice for sensitive reagents.

This streamlined methodology was inventive as it enabled direct amidation of 7-D-Glucuronic acid-5,6-dihydroxyflavone without any protecting groups, using mild, biocompatible reagents in a green hydroalcoholic medium, thereby preventing thermal or hydrolytic degradation of the sensitive 7-D-Glucuronic acid-5,6-dihydroxyflavone scaffold. The approach provided activation, coupling, precipitation-based isolation, and purification in a single vessel, significantly reducing solvent use, processing time, and manufacturing cost compared to conventional multi-step derivatization methods.

Compound of formula Ia (¹H NMR, 400 MHz, DMSO-d₆): δ 12.10 (br s, 1H), 9.35 (br s, 1H), 8.85 (br s, 1H), 7.95 (d, J=8.8 Hz, 1H), 7.65 (d, J=2.2 Hz, 1H), 7.50 (dd, J=8.8, 2.2 Hz, 1H), 6.95 (s, 1H), 5.20 (d, J=7.8 Hz, 1H), 4.20-3.60 (m, 5H), 3.75 (s, 3H), 3.40 (dd, J=9.6, 4.8 Hz, 1H), 1.30 (d, J=7.0 Hz, 3H); Compound of formula Ib (¹H NMR, 400 MHz, DMSO-d₆): δ 12.12 (br s, 1H), 9.32 (br s, 1H), 8.82 (br s, 1H), 7.90 (d, J=8.6 Hz, 1H), 7.60 (d, J=2.1 Hz, 1H), 7.45 (dd, J=8.6, 2.1 Hz, 1H), 6.92 (s, 1H), 5.18 (d, J=7.8 Hz, 1H), 4.18-3.58 (m, 5H), 3.87 (s, 3H), 3.75 (s, 3H), 3.38 (dd, J=9.4, 4.6 Hz, 1H), 1.28 (d, J=7.0 Hz, 3H); Compound of formula-Ic (¹H NMR, 400 MHz, DMSO-d₆): δ 12.08 (br s, 1H), 9.30 (br s, 1H), 8.80 (br s, 1H), 7.88 (d, J=8.7 Hz, 1H), 7.62 (d, J=2.2 Hz, 1H), 7.48 (dd, J=8.7, 2.2 Hz, 1H), 6.90 (s, 1H), 5.17 (d, J=7.8 Hz, 1H), 4.16-3.55 (m, 5H), 3.92 (s, 3H), 3.75 (s, 3H), 3.36 (dd, J=9.5, 4.5 Hz, 1H), 1.26 (d, J=7.0 Hz, 3H).

### HPLC Analytical Method:

The purity of Compounds of Formula I were determined by High-Performance Liquid Chromatography (HPLC) using a C18 reverse-phase column (250×4.6 mm, 5 m). The mobile phase consisted of Solvent A (water containing 0.10 TFA) and Solvent B (acetonitrile containing 0.1% TFA). A gradient elution of 10-60% Solvent B over 20 minutes was employed at a flow rate of 1.0 mL/min. The column was maintained at 30° C. and UV detection was set at 254 nm. The sample was prepared in the mobile phase, filtered through a 0.45 m membrane, and injected into the system. Under these conditions, Compound of formula Ia eluted with a retention time of approximately 9.5 minutes and consistently showed a purity of ≥980% (area %).

### Example 2

Composition 1- Tablet/Capsule

| Ingredient | mg per unit dose |
|---|---|
| Methyl 2-((2S,3S,4S,5R,6S)-6-((5,6- | 200 |
| dihydroxy-4-oxo-2-phenyl-4H-chromen- | |
| 7-yl)oxy)-3,4,5-trihydroxytetrahydro- | |
| 2H-pyran-2-carboxamido)propanoate | |
| Magnesium Stearate | 0.1-10 |
| Hydroxypropyl Methylcellulose | 0.5-10 |
| Microcrystalline Cellulose | 0.1-10 |
| Polyvinylpyrrolidone | 0.1-10 |
| Pregelatinized Starch | 0.1-5 |
| Talc | 0.1-5 |
| Mannitol | 0.1-2 |
| Propylene Glycol | QS |
| Water | QS |
| Average weight | 210-280 mg |

Composition 2: Tablet/Capsule

| Ingredient | mg per unit dose |
|---|---|
| Methyl 2-((2S,3S,4S,5R,6S)-6-((5-methoxy,6- | 100 |
| hydroxy-4-oxo-2-phenyl-4H-chromen-7- | |
| yl)oxy)-3,4,5-trihydroxytetrahydro- | |
| 2H-pyran-2-carboxamido)propanoate; | |
| Magnesium Stearate | 0.1-5 |
| Ascorbic acid | 0.1-5 |
| Colloidal Silicon dioxide | 0.1-5 |
| Hydroxypropyl Methylcellulose | 0.1-5 |
| Polyvinylpolypyrrolidone | 0.1-5 |
| Pregelatinized Starch | 0.1-10 |
| Croscarmellose Sodium | 0.1-5 |
| Talc | 0.1-5 |
| Polysorbate 80 | 0.1-5 |
| Mannitol | 0.1-1 |
| Water | QS |
| Average weight | 110-160 mg |

Composition 3: Tablet/Capsule

| Ingredient | mg per unit dose |
|---|---|
| Methyl 2-((2S,3S,4S,5R,6S)-6-((5-hydroxy,6- | 100 |
| methoxy-4-oxo-2-phenyl-4H-chromen-7- | |
| yl)oxy)-3,4,5-trihydroxytetrahydro- | |
| 2H-pyran-2-carboxamido) propanoate | |
| or combination thereof. | |
| Microcrystalline Cellulose | 0.1-10 |
| Silicon dioxide | 0.1-5 |
| Hydroxypropyl Methylcellulose | 0.1-5 |
| Stearic acid | 0.1-5 |
| Pregelatinized starch | 0.1-10 |
| Talc | 0.1-5 |
| Polysorbate 80 | 0.1-5 |
| Polyethylene glycol | QS |
| Water | QS |
| Average weight | 110-150 mg |

### Example 3: Quantification of EPAC2 Expression in Treatment with Test Compounds by ELISA Test

EPAC2 levels were quantified by a commercial sandwich ELISA using a standardized workflow adapted from the standard procedure.

Rat ventricular myocytes (cardiac cells) were homogenized on ice in chilled RIPA/IP extraction buffer containing protease inhibitors, and the lysates were clarified by centrifugation (12,000-14,000×g, 10-15 min, 4° C.). Total protein in the supernatant was quantified by BCA to enable normalization. Pilot dilutions were performed to ensure that the sample absorbance values fell within the standard curve range of the EPAC2 ELISA kit. Diluted samples (100 μL/well), along with test samples and controls, were loaded in triplicate into antibody-coated wells and incubated at room temperature for 1-2 h for antigen binding. The wells were then washed 3-5 times with PBS containing 0.05% Tween-20, followed by incubation with biotinylated detection antibody for 1 h. After an additional wash cycle, streptavidin—HRP was added and incubated for 20-30 min, followed by five washes and the addition of TMB substrate, which was allowed to develop in the dark for 5-15 min. The reaction was stopped with acid, and absorbance was measured at 450 nm (reference 620-650 nm). EPAC2 concentrations were determined from a 4-parameter logistic standard curve, corrected for sample dilution, and normalized to protein content (expressed as ng EPAC2 per mg total protein). All assays met acceptance criteria; Statistical analysis was performed using one-way ANOVA followed by Dunnett's post-hoc test versus control (n=3).

The table below reports representative results (mean±SEM, n=3) for Control, Non-Conjugated-Free base, Compounds of formula Ia, Ib, Ic. OD values were measured at 450 nm and converted to normalized EPAC2 (ng EPAC2/mg total protein) using the kit standard curve; fold-change is relative to Control and p-values are from Dunnett's multiple-comparison test versus Control.

| | Mean OD | EPAC2 | Fold vs | p-value vs |
|---|---|---|---|---|
| Group | [450 nm] | (ng/mg) | Control | Control |
| Control (vehicle) | 0.150 ± 0.005 | 0.10 ± 0.01 | 1.00 | — |
| (Non- Conjugated) | 0.195 ± 0.008 | 0.13 ± 0.02 | 1.30 | p = 0.043 |
| 7-D-Glucuronic | | | | |
| acid-5,6- | | | | |
| dihydroxyflavone | | | | |
| Compound of | 0.300 ± 0.010 | 0.23 ± 0.02 | 2.20 | p = 0.0012 |
| formula Ia | | | | |
| Methyl 2- | | | | |
| ((2S,3S,4S,5R,6S)-6- | | | | |
| ((5,6-dihydroxy-4-oxo-2- | | | | |
| phenyl-4H-chromen-7- | | | | |
| yl) oxy)-3,4,5- | | | | |
| trihydroxytetrahydro-2H- | | | | |
| pyran-2-carboxamido) | | | | |
| propanoate | | | | |
| Compound of | 0.305 ± 0.012 | 0.21 ± 0.02 | 2.10 | p = 0.0011 |
| formula Ib | | | | |
| Methyl 2- | | | | |
| ((2S,3S,4S,5R,6S)-6-((5- | | | | |
| methoxy,6-hydroxy-4- | | | | |
| oxo-2-phenyl-4H- | | | | |
| chromen-7-yl)oxy)-3,4,5- | | | | |
| trihydroxytetrahydro-2H- | | | | |
| pyran-2- | | | | |
| carboxamido)propanoate; | | | | |
| Compound of | 0.315 ± 0.015 | 0.20 ± 0.02 | 2.20 | p = 0.0015 |
| formula Ic | | | | |
| Methyl 2- | | | | |
| ((2S,3S,4S,5R,6S)-6-((5- | | | | |
| hydroxy,6-methoxy-4- | | | | |
| oxo-2-phenyl-4H- | | | | |
| chromen-7-yl)oxy)-3,4,5- | | | | |
| trihydroxytetrahydro-2H- | | | | |
| pyran-2-carboxamido) | | | | |
| propanoate. | | | | |

We claim:

1. Carboxamide compounds or a pharmaceutically acceptable salt thereof for activating Exchange Protein Activated by cAMP (EPAC2) expression, having a Formula-I: 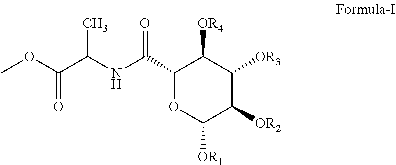
wherein R₁, R₂, R₃ and R₄ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, and a 5 or 6-membered heterocyclic ring having 1 to 4 heteroatoms, wherein the heteroatoms are selected from O, N, and S, wherein the 5 or 6-membered heterocyclic ring is, 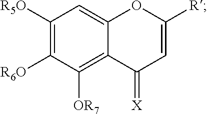
wherein R′ is selected from the group consisting of hydrogen, hydroxyl, halogen, (C₁-C₆) alkyl, (C₂-C₈) alkylene, (C₂-C₈) alkyne and (C₆-C₁₀) aryl;
wherein R₅, R₆ and R₇ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, a 5 or 6-membered heterocyclic ring having 1 to 4 heteroatoms, —C═O, —C═OR₄, —CO₂R₅, —NO₂, —CONR₅R₆, —N(R₅)COR₆; —NR₅R₆, —N(R₅)COR₆, —CN, —CHFCN and CF₂CN; wherein X is substituted or unsubstituted 0, N and S.

2. The carboxamide compounds of Formula-I as claimed in claim 1, wherein the carboxamide compound is selected from the group consisting of:

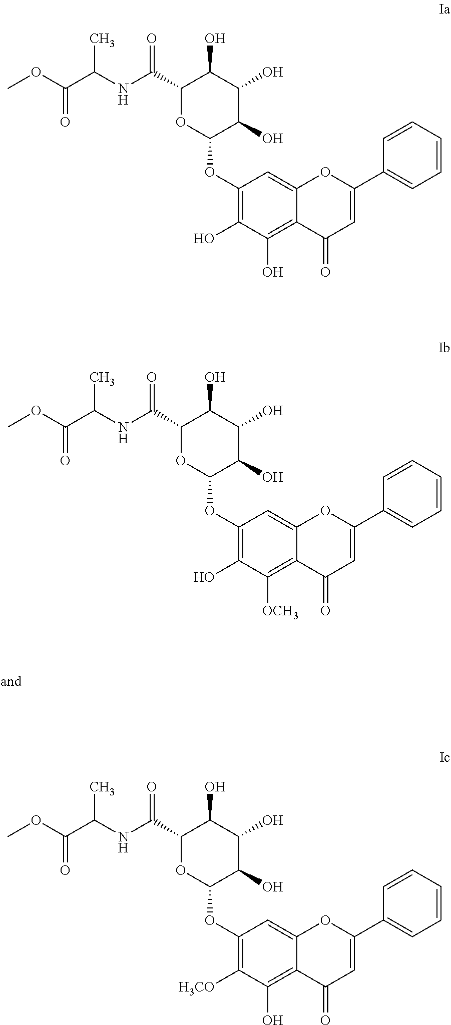 [SMILES: COC(=O)C(C)NC(=O)[C@H]1O[C@@H](Oc2cc3oc(-c4ccccc4)cc(=O)c3c(O)c2O)[C@H](O)[C@@H](O)[C@@H]1O ; COC(=O)C(C)NC(=O)[C@H]1O[C@@H](Oc2cc3oc(-c4ccccc4)cc(=O)c3c(OC)c2O)[C@H](O)[C@@H](O)[C@@H]1O ; COC(=O)C(C)NC(=O)[C@H]1O[C@@H](Oc2cc3oc(-c4ccccc4)cc(=O)c3c(O)c2OC)[C@H](O)[C@@H](O)[C@@H]1O]

3. The carboxamide compounds of Formula-I as claimed in claim 1, wherein the carboxamide compounds are selected from the group consisting of methyl 2-((2S,3S,4S,5R,6S)-6-((5,6-dihydroxy-4-oxo-2-phenyl-4H-chromen-7-yl)oxy)-3,4,5-trihydroxytetrahydro-2H-pyran-2-carboxamido)propanoate; methyl 2-((2S,3S,4S,5R,6S)-6-((5-methoxy,6-hydroxy-4-oxo-2-phenyl-4H-chromen-7-yl)oxy)-3,4,5-trihydroxytetrahydro-2H-pyran-2-carboxamido)propanoate; methyl 2-((2S,3S,4S,5R,6S)-6-((5-hydroxy,6-methoxy-4-oxo-2-phenyl-4H-chromen-7-yl)oxy)-3,4,5-trihydroxytetrahydro-2H-pyran-2-carboxamido) propanoate, and a combination thereof.

4. A one-pot process for preparing a carboxamide compound of Formula-I, the process comprising:
a) dissolving a 7-D-Glucuronic acid-5,6-dihydroxyflavone compound in a hydroalcoholic solvent;
b) activating a carboxyl group of the 7-D-Glucuronic acid-5,6-dihydroxyflavone compound in-situ using a carbodiimide coupling reagent and N-hydroxysuccinimide at a temperature of below 5° C. to form an activated intermediate;
(c) reacting the activated intermediate with methyl alaninate in presence of an organic base to form a product;
(d) precipitating the product by solvent adjustment; and, wherein Formula I is 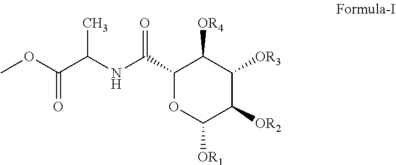
wherein R₁, R₂, R₃ and R₄ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, and a 5 or 6-membered heterocyclic ring having 1 to 4 heteroatoms, wherein the heteroatoms are selected from O, N, and S, wherein the 5 or 6-membered heterocyclic ring is, 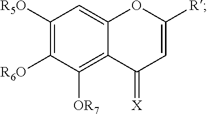
wherein R′ is selected from the group consisting of hydrogen, hydroxyl, halogen, (C₁-C₆) alkyl, (C₂-C₈) alkylene, (C₂-C₈) alkyne and (C₆-C₁₀) aryl;
wherein R₅, R₆ and R₇ are similar or independently selected from the group consisting of hydrogen, hydroxyl, halogen, (C₁-C₆) alkyl, (C₂-C₅) alkylene, (C₂-C₈) alkyne, (C₃-C₅) cycloalkyl, (C₁-C₆) alkoxy, (C₆-C₁₀) aryl, a 5 or 6-membered heterocyclic ring having 1 to 4 heteroatoms, —C═O, —C═OR₄, —CO₂R₅, —NO₂, —CONR₅R₆, —N(R₅)COR₆; —NR₅R₆, —N(R₅)COR₆, —CN, —CHFCN and CF₂CN; wherein X is substituted or unsubstituted 0, N and S.

5. The one-pot process as claimed in claim 4, wherein the hydroalcoholic solvent comprises a C₁-C₄ alkanol and water.

6. The one-pot process as claimed in claim 4, wherein the organic base is selected from the group consisting of triethylamine, N,N-diisopropylethylamine, N-methylmorpholine, pyridine, sodium, potassium hydroxide, ammonium hydroxide, ammonia, primary or secondary amine, and mixtures thereof.

7. A pharmaceutical composition comprising a therapeutically effective amount of a carboxamide compound of Formula-I and pharmaceutically acceptable excipients, wherein the excipients comprise a diluent, a binder, a disintegrant, a lubricant, a glidant, a solubilizing agent or a surfactant, a stabilizer, a plasticizer, an antioxidant, and an additive, wherein Formula I is 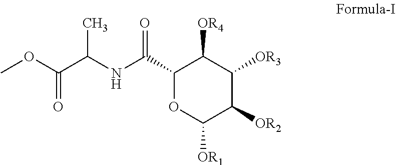
wherein R₁, R₂, R₃ and R₄ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, and a 5 or 6-membered heterocyclic ring having 1 to 4 heteroatoms, wherein the heteroatoms are selected from O, N, and S, wherein the 5 or 6-membered heterocyclic ring is, 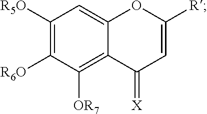
wherein R′ is selected from the group consisting of hydrogen, hydroxyl, halogen, (C₁-C₆) alkyl, (C₂-C₈) alkylene, (C₂-C₈) alkyne and (C₆-C₁₀) aryl;
wherein R₅, R₆ and R₇ are similar or independently selected from the group consisting of hydrogen, hydroxyl, halogen, (C₁-C₆) alkyl, (C₂-C₈) alkylene, (C₂-C₈) alkyne, (C₃-C₈) cycloalkyl, (C₁-C₆) alkoxy, (C₆-C₁₀) aryl, a 5 or 6-membered heterocyclic ring having 1 to 4 heteroatoms, —C═O, —C═OR₄, —CO₂R₅, —NO₂, —CONR₅R₆, —N(R₅)COR₆; —NR₅R₆, —N(R₅)COR₆, —CN, —CHFCN and CF₂CN; wherein X is substituted or unsubstituted 0, N and S.

8. The pharmaceutical composition as claimed in claim 7, wherein the diluent is present in a range of 1 to 30%; the binder is present in a range of 0.1 to 25%; the disintegrant is present in a range of 0.1 to 10%; the lubricant is present in a range of 0.1 to 10.0%; the glidant is present in a range of 0.1 to 5.0%; the solubilizing agent or the surfactant is present in a range of 0.1 to 5.0%; the stabilizer is present in a range of 0.1 to 5.0%; the plasticizer is present in a range of 0.1 to 5.0%; the antioxidant is present in a range of 0.1 to 10%; the additive is present in a range of 1 to 20% by weight of total pharmaceutical composition.

9. The pharmaceutical composition as claimed in claim 7, wherein the pharmaceutical composition is characterized for treating or preventing neurological disorders associated with impaired EPAC2 activity, wherein the neurological disorders comprise cognitive impairment, synaptic dysfunction, Alzheimer's disease, Parkinson's disease, or neurodegeneration.

10. The pharmaceutical composition as claimed in claim 7, wherein a daily dose of the pharmaceutical composition ranges from about 0.1 mg per day to about 5000 mg per day.
